SEQUENCE LISTING (9 sequences)

SEQ ID NO: 1 - protein, 240 aa, Artificial sequence
  Synthetic polypeptide
MSKGEELFTGVVPILVELDGDVNGHKFSVSGEGEGDATYGKLTLKLLCTTGKLPVPWPTLVTTLGYGVQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSALFKDPNEKRDHMVLLEFLTAAGITEGMNELYKEL

SEQ ID NO: 2 - protein, 228 aa, Artificial sequence
  Synthetic polypeptide
MVSKGEELFTGVVPILVELDGDVNGHRFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSALSKDPNEKRDHMVLLEFVTAA

SEQ ID NO: 3 - protein, 481 aa, Artificial sequence
  Synthetic polypeptide
MSKGEELFTGVVPILVELDGDVNGHKFSVSGEGEGDATYGKLTLKLLCTTGKLPVPWPTLVTTLGYGVQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSALFKDPNEKRDHMVLLEFLTAAGITEGMNELYKELCPKESCNLFVLKDMVSKGEELFTGVVPILVELDGDVNGHRFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSALSKDPNEKRDHMVLLEFVTAA

SEQ ID NO: 4 - protein, 7 aa, Artificial sequence
  Synthetic peptide
PLGVYAR

SEQ ID NO: 5 - protein, 13 aa, Artificial sequence
  Synthetic peptide
CPKESCNLFVLKD

SEQ ID NO: 6 - protein, 8 aa, Artificial sequence
  Synthetic peptide
AAQNLYEK

SEQ ID NO: 7 - protein, 8 aa, Artificial sequence
  Synthetic peptide
NFAAQMAG

SEQ ID NO: 8 - protein, 8 aa, Artificial sequence
  Synthetic peptide
KPNMIDAA

SEQ ID NO: 9 - protein, 14 aa, Artificial sequence
  Synthetic peptide
KKKKKKSKTKCVIM

(19) United States
(12) Patent Application Publication
(10) Pub. No.: US 2011/0171675 A1
(43) Pub. Date: Jul. 14, 2011

(54) FRET-BASED MEMBRANE-TYPE 1 MATRIX METALLOPROTEINASE BIOSENSOR AND METHODS FOR USING THE SAME

(76) Inventors: Yingxiao Wang, Champaign, IL (US); Shaoying Lu, Champaign, IL (US); Mingxing Ouyang, Urbana, IL (US)

(21) Appl. No.: 13/057,488

(22) PCT Filed: Aug. 4, 2009
(86) PCT No.: PCT/US09/52713

§ 371 Date: Mar. 29, 2011

Related U.S. Application Data
(60) Provisional application No. 61/085,912, filed on Aug. 4, 2008.

(51) Int. Cl. C12Q 1/37; C12N 9/96; C07H 21/04; C12N 15/63; C12N 5/10

(52) U.S. Cl. 435/23; 435/188; 536/23.2; 435/320.1; 435/325

(57) ABSTRACT

The present invention is a novel FRET-based biosensor composed of ECFP and YPet fluorescent proteins operably linked via a MT1-MMP recognition sequence for use in the detection of cancer cells in a biological sample.

This application is claims the benefit of priority of U.S. Provisional Application No. 61/085,912, filed Aug. 4, 2008, the content of which is incorporated herein by reference in its entirety.

### BACKGROUND OF THE INVENTION

Breast cancer patients have been conventionally diagnosed via mammography. However, in contrast to breast cancer diagnostics, existing prognostic methods are neither specific nor sensitive. For example, nearly 30% of patients with node-negative breast cancer will show metastasis, while 40% of the patients with breast cancer that spreads to axillary lymph nodes can survive for 10 years without recurrence. Recent evidence suggests that circulating tumor cells (CTCs) can serve as an independent prognostic factor for breast cancer metastasis (Cristofanilli, et al. (2004) N. Engl. J. Med. 351:781-791; Xenidis, et al. (2006) J. Clin. Oncol. 24:3756-3762). CTCs are identified based upon the absence of CD45 and high level of cytokeratin expression (Cristofanilli, et al. (2004) supra). Breast cancer patients are considered to be CTC positive with aggressive cancer if there have more than five CTCs in a 7.5 ml blood sample.

Although counting CTCs is considered a breakthrough in monitoring the metastatic potential of breast cancer, this test is unsatisfactory in accuracy. In fact, it has been reported that not all the CTCs are malignant, although the majority of these cells in peripheral blood is disseminated from tumors (Fehm, et al. (2002) Clin. Cancer Res. 8:2073-2084).

### SUMMARY OF THE INVENTION

The present invention is a biosensor composed of Enhanced Cyan Fluorescent Protein (ECFP) and Yellow fluorescent Protein for Energy Transfer (YPet) operably linked via an MT1-MMP recognition sequence, wherein said biosensor has a dynamic range of 50% in vivo. In one embodiment, the biosensor further includes a positively charged tag, e.g., a 5 to 30 amino acid residue oligopeptide composed of arginine or histidine. In another embodiment, the MT1-MMP recognition sequence is a 7 to 20 amino acid residue oligopeptide, e.g., as set forth in SEQ ID NO:4, SEQ ID NO:5, SEQ ID NO:6, SEQ ID NO:7, or SEQ ID NO:8. A kit and an isolated chimeric nucleic acid molecule encoding the biosensor of the invention are also provided as is an expression vector and host containing the same.

The present invention also features a method for using this biosensor to detect a cancer cell in a biological sample such as blood. In certain embodiments, the cancer cell is a circulating tumor cell of metastatic colorectal cancer, breast cancer or prostate cancer.

### BRIEF DESCRIPTION OF THE DRAWINGS

FIG. 1 is a schematic drawing showing an MT1-MMP biosensor tagged with nine arginine residues localized to the cell surface. FIG. 1A depicts normal cells, wherein the biosensor is intact and displays high FRET. FIG. 1B depicts cancer cells wherein the biosensor is cleaved by active MT1-MMP and displays low FRET.

FIG. 2 depicts MT1-MMP biosensor tethered to the plasma membrane (PM) via a transmembrane domain (TM).

### DETAILED DESCRIPTION OF THE INVENTION

MT1-MMP is a key enzyme in tumor cell invasion and metastasis, including breast cancer (Deryugina & Quigley (2006) Cancer Metastasis Rev. 25:9-34; Munoz-Najar, et al. (2006) Oncogene 25:2379-2392). A fluorescence resonant energy transfer (FRET)-based ratio metric biosensor has now been developed to detect the activity of MT1-MMP in live cells. Evidence demonstrates that this biosensor is sensitive and specific in reporting MT1-MMP activities. The application of this biosensor to detect circulating tumor cells allows for convenient, specific, and reliable prognosis of breast cancer using blood samples. This biosensor can be used in methods to predict disease-free survival time and overall survival rate of cancer patients. It can also be used in methods for assessing the effectiveness of therapy by monitoring the number of aggressive circulating tumor cells before and after therapeutic treatment. This can facilitate the design and implementation of personalized cancer therapy for individual patients. This novel detection method can be performed alone or in combination with a standard circulating tumor cell test to improve the accuracy of the prognostic tests for breast cancer metastasis. In addition, because of the general high activity of MT1-MMP in many cancer types, the MT1-MMP FRET-based assay can be generalized and applied to detect circulating tumor cells in other cancer patients, such as metastatic colorectal cancer or metastatic prostate cancer.

Accordingly, the present invention features a FRET-based biosensor with an MT1-MMP recognition sequence for the detection of tumor cells. As is conventional in the art, FRET occurs when two fluorophores are in proximity, with the emission spectrum of the donor overlapping the excitation spectrum of the acceptor. Once the two proteins of the FRET pair are separated by a sufficient distance, FRET does not occur. The present FRET-based sensor employs Enhanced Cyan Fluorescent Protein (ECFP) and Yellow fluorescent Protein for Energy Transfer (YPet), which have been found to significantly enhance the dynamic range of FRET in comparison to the conventional FRET pairs such as ECFP and EYFP variants (Citrine or Venus). Accordingly, the biosensor of the invention is a MT1-MMP recognition sequence flanked on either side by ECFP or YPet; i.e., one fluorescent protein on one side and the other fluorescent protein on the other side.

ECFP and YPet proteins of use in accordance with the present invention are known in the art. For example, a suitable ECFP protein is described in GENBANK Accession No. AAX29988. An exemplary ECFP protein is set forth in SEQ ID NO:1. Similarly, a suitable YPet protein of use in this invention is described by Nguyen & Daugherty ((2005) Nat. Biotechnol. 23(3):355-60). An exemplary YPet protein is set forth in SEQ ID NO:2.

An MT1-MMP recognition sequence is an oligopeptide substrate, which is cleaved by MT1-MMP. Examples of MT1-MMP recognition sequences are listed in Table 1. Other suitable MT1-MMP recognition sequences are known in the art and a comprehensive list of extracellular proteases and their cognate substrate peptides is available at the MEROPS database (see Rawlings, et al. (2002) Nucl. Acids Res. 30:343-346).

TABLE 1

| | SEQ ID |
|---|---|
| Recognition Sequence | NO: |
| Pro-Leu-Gly-Val-Tyr-Ala-Arg¹ | 4 |
| Cys-Pro-Lys-Glu-Ser-Cys-Asn-Leu-Phe-Val- | 5 |
| Leu-Lys-Asp | |
| Ala-Ala-Gln-Asn-Leu-Tyr-Glu-Lys² | 6 |
| Asn-Phe-Ala-Ala-Gln-Met-Ala-Gly³ | 7 |
| Lys-Pro-Asn-Met-Ile-Asp-Ala-Ala⁴ | 8 |

¹Iemura, et al. (2004) Pept. Sci. 2003: 339-342.
²Kim, et al. (2006) Biochem. Biophys. Res. Commun. 339:47-54.
³Fukui, et al. (2002) J. Biol. Chem. 277:2193-2201.
⁴Hiller, et al. (2000) J. Biol. Chem. 275:33008-33013.

The biosensor of the present invention can be assembled with either the ECFP or YPet protein at N-terminus, so long as the MT1-MMP recognition sequence is operably linked or located between the ECFP and YPet proteins and ECFP, YPet, and the MT1-MMP recognition sequence are in-frame to produce a contiguous fusion protein. The ECFP and YPet are operably linked via the MT1-MMP recognition sequence in the sense that when the MT1-MMP recognition sequence is present between the ECFP and YPet proteins, FRET occurs upon excitation of the donor protein. Because FRET is a distance-dependent interaction, an appropriate distance for the donor and acceptor FRET pair is typically in the range of 10-60 Å. Thus, in some embodiments, the MT1-MMP recognition sequence is a 4-50 amino acid residue oligopeptide. In other embodiments, the MT1-MMP recognition sequence is a 7-20 amino acid residue oligopeptide. An exemplary biosensor protein of this invention is set forth in SEQ ID NO:3.

Advantageously, the biosensor of the present invention has a dynamic range, i.e., total “spread” of signal change before and after cleavage of the MT1-MMP recognition sequence, exceeding 100%. Indeed, when purified, the biosensor of the invention has a dynamic range of %570, and when used in vivo, the dynamic range is 50%. Accordingly, in some embodiments, the dynamic range of the biosensor of the invention is in the range of 40% to 600% depending on the application and at least 50% in vivo.

The biosensor of the invention can be produced by recombinant DNA technology or chemical synthesis, or a combination thereof. Recombinant production is particularly suitable to producing large quantities of the biosensor. In this regard, nucleic acid molecules encoding the ECFP and YPet proteins and the MT1-MMP recognition sequence are ligated together in-frame in accordance with conventional recombinant DNA techniques and cloned into an expression vectors. Alternatively, the chimeric nucleic acid molecule can be synthesized by conventional techniques including automated DNA synthesis or polymerase chain reaction (PCR) amplification. PCR amplification of gene fragments can be carried out using anchor primers which give rise to complementary overhangs between two consecutive gene fragments which are subsequently annealed and reamplified to generate a chimeric gene sequence (see, e.g., Current Protocols in Molecular Biology, eds. Ausubel, et al. John Wiley & Sons, 1992). When the MT1-MMP recognition sequence is a short peptide, nucleic acids encoding the peptide can be incorporated into the 5′ or 3′ anchor primers used to amplify nucleic acids encoding one or both of the fluorescent proteins (e.g., using add-on PCR). The fusion protein is then produced by introducing the chimeric nucleic acid molecule into an expression vector and using a suitable host cell to transcribe and translate the fusion protein.

Construction of expression vectors and recombinant production from the appropriate DNA molecules are performed by methods known in the art per se. Expression vectors and host cells can be from any suitable prokaryotic or eukaryotic system. Prokaryotes most frequently are represented by various strains of E. coli. However, other microbial strains can also be used, such as bacilli, for example Bacillus subtilis, various species of Pseudomonas, or other bacterial strains. In such prokaryotic systems, plasmid vectors which contain replication sites and control sequences derived from a species compatible with the host are used. For example, E. coli is typically transformed using derivatives of pBR322, a plasmid derived from an E. coli species discussed by Bolivar, et al. ((1977) Gene 2:95). Commonly used prokaryotic control sequences, which are defined herein to include promoters for transcription initiation, optionally with an operator, along with ribosome binding site sequences, including such commonly used promoters as the beta-lactamase (penicillinase) and lactose (lac) promoter systems (Change, et al. (1977) Nature 198:1056) and the tryptophan (trp) promoter system (Goeddel, et al. (1980) Nucleic Acids Res. 8:4057) and the lambda-derived P_{L} promoter and N-gene ribosome binding site (Shimatake, et al. (1981) Nature 292:128). Any available promoter system compatible with prokaryotes can be used.

Expression systems useful in the eukaryotic hosts generally include promoters derived from appropriate eukaryotic genes. A class of promoters useful in yeast includes, for example, promoters for synthesis of glycolytic enzymes, including those for 3-phosphoglycerate kinase (Hitzeman, et al. ((1980) J. Biol. Chem. 255:2073). Other promoters include those from the enolase gene (Holland, et al. (1981) J. Biol. Chem. 256:1385) or the Leu2 gene obtained from YEp13 (Broach, et al. (1978) Gene 8:121).

Suitable mammalian promoters include the early and late promoters from SV40 (Fiers, et al. (1978) Nature 273:113) or other viral promoters such as those derived from polyoma, adenovirus II, bovine papilloma virus or avian sarcoma viruses.

The expression system is constructed from the foregoing control elements operably linked to nucleic acids encoding the biosensor disclosed herein using standard methods, employing standard ligation and restriction techniques which are well-understood in the art. Isolated plasmids, DNA sequences, or synthesized oligonucleotides are cleaved, tailored, and relegated in the form desired.

Correct ligation during plasmid construction can be confirmed by first transforming a suitable E. coli strain with the ligation mixture. Successful transformants are selected using a conventional selectable marker, e.g., ampicillin, tetracycline or other antibiotic resistance or using other markers depending on the mode of plasmid construction, as is understood in the art. Plasmid from the transformants can then be prepared according to conventional methods. See, e.g., the method of Clewell, et al. (1969) Proc. Natl. Acad. Sci. USA 62:1159. The isolated DNA is analyzed by restriction and/or sequenced according to standard laboratory practices.

The constructed vector is then transformed into a suitable host cell for production of the biosensor. Depending on the host cell used, transformation is done using standard techniques appropriate to such cells. Calcium treatment employing calcium chloride, as described by Cohen ((1972) Proc. Natl. Acad. Sci. USA 69:2110), or the RbC1 method described in Maniatis, et al. ((1982) Molecular Cloning: A Laboratory Manual, Cold Spring Harbor Press, p. 254) can be used for prokaryotes or other cells which contain substantial cell wall barriers. For mammalian cells without such cell walls, the calcium phosphate precipitation method of Graham and van der Eb, ((1978) Virology 52:546) or electroporation is preferred. Transformations into yeast can be carried out according to the method of Van Solingen, et al. ((1978) J. Bacter. 130:946) or Hsiao, et al. ((1979) Proc. Natl. Acad. Sci. USA 76:3829.

The transformed host cells are then cultured under conditions favoring expression of the biosensor and the recombinantly produced protein recovered from the cells or cell supernatant. To facilitate purification, one or more purification tags can be incorporated into the biosensors of the invention. Exemplary tags include, e.g., FLAG, c-myc and the like.

In particular embodiments of this invention, the biosensor further includes a positively charged tag. A positively charged tag is molecule that has a net positive charge, wherein the sum of the charges present under the desired reaction conditions is +1 or greater. In this regard, a molecule having a net positive charge would migrate toward the negative electrode in an electrical field. As used herein, a tag refers to a molecule that is attached to the N- or C-terminus of the instant biosensor. When the tag is composed of amino acid residues, the tag can be attached as a fusion protein. By way of exemplification, a positively charged tag is a positively charged oligopeptide (e.g., 5 to 30 amino acid residues) containing one or more arginines and/or histidines. In particular embodiments, the positively charged tag is an oligopeptide composed of arginines.

For use in this invention, the instant biosensor can be use provided in a kit. As used herein, the term “kit” refers to any delivery system for delivering materials. In the context of assays, such delivery systems include systems that allow for the storage, transport, or delivery of reagents (e.g., substrates, enzymes, etc. in the appropriate containers) and/or supporting materials (e.g., buffers, written instructions for performing the assay, etc.) from one location to another. For example, kits include one or more enclosures (e.g., boxes) containing the relevant reaction reagents and/or supporting materials. It is contemplated that a kit of the present invention would include at least two containers, one with biosensor of the invention and another with assay buffer. Detailed instruction of use and the optimal biosensor concentration and incubation time will also be included in the kit. Instructions for using the kit would indicate, e.g., that blood cells be centrifuged, washed, and incubated with the biosensor and buffer at 37° C. to facilitate the cleavage of the biosensor and amplify FRET signal before being subjected to flow cytometry screening. Such a kit is expected to provide an easy, efficient and accurate counting of malignant CTCs in a blood sample. This product can be used alone or in combination with a standard CTC test to measure the number of circulating tumor cells in blood samples from patients.

Having demonstrated that the biosensor of the present invention can distinguish a cancer cell from a normal cell, the present invention also features a method for detecting a cancer cell in a biological sample. The method involves contacting a biological sample with the biosensor of the invention and detecting FRET, wherein the level of FRET is indicative of the presence of a cancer cell. A biological sample in accordance with this method of the invention can be a biopsy sample, tissue sample, or fluid sample such as urine, sputum, blood, peritoneal fluid, ascites fluid or the like. In particular embodiments, the biological sample is blood. In this regard, the present invention embraces the detection circulating tumor cells.

Detection of the level of FRET (i.e., the ECFP:YPet emission ratios) can be determined using any conventional fluorescent-based detection system including, for example, a fluorometer or flow cytometry. When employing such systems, the level of FRET can be determined based upon a comparison with one or more control samples, e.g., a sample known to contain cancer cells or normal healthy cells. Alternatively, the level of FRET can be based upon an experimentally determined threshold, wherein levels above or below the threshold level are indicative of normal cells or cancer cells. In so far as MT1-MMP cleaves the MT1-MMP recognition sequence of the biosensor of the invention, a decrease in FRET is indicative of MT1-MMP activity, wherein cancer cells expressing high levels of MT1-MMP decrease FRET as compared to normal healthy cells. Cancer cell detection can be used to facilitate both the diagnosis and prognosis of a variety of cancers including metastatic breast, colorectal, and prostate cancers.

The FRET-based biosensor of this invention also finds application in visualizing MT1-MMP activity with high spatiotemporal resolution in live cells and advancing the understanding of MT1-MMP in cancer development. Furthermore, this biosensor can be used as a readout indicator for the high-throughput screening of inhibitors for MT1-MMP and its associated diseases, including cancer. Therefore, the biosensor of the invention is of use in the fundamental biological study of cancers and related diseases in which MT1-MMP plays a critical role.

The invention is described in greater detail by the following non-limiting examples.

### Example 1

### FRET Biosensor Capable of Detecting MT1-MMP

### Activity

Using ECFP and YPet, an improved version of EYFP, a MT1-MMP biosensor was developed. The MT1-MMP biosensor was composed of an N-terminal YPet as the FRET acceptor; a Sac' restriction enzyme site; a MT1-MMP recognition sequence derived from MMP-2 (Cys-Pro-Lys-Glu-Ser-Cys-Asn-Leu-Phe-Val-Leu-Lys-Asp; SEQ ID NO:5), with amino acid residues Asn-Leu cleavable by MT1-MMP; and a C-terminal ECFP as the FRET donor.

It was determined whether MT1-MMP could cause a FRET change of the biosensor. The incubation of the purified biosensor protein with the catalytic domain of MT1-MMP caused a significant decrease in YPet spectrum max (526 nm) and a concomitant increase in ECFP spectrum max (476 nm), a clear indication of FRET decrease. With gel electrophoresis analysis and in vitro FRET assay, it was confirmed that the catalytic domain of MT1-MMP cleaved the wild-type MT1-MMP biosensor but not a biosensor with a mutated protease recognition sequence.

### Example 2

### Spectral Properties of Purified FRET Biosensors

The bottleneck of genetically-encoded FRET biosensors based on FPs is their poor dynamic ranges. CyPet, a variant of ECFP was developed for high-efficiency FRET, however, this protein folds poorly at 37° C. and is not suitable for live cell imaging. To develop a new FRET pair for live cell imaging, Citrine in the Src FRET biosensor was replaced with YPet. The resulting FRET pair was composed of YPet and ECFP. An in vitro kinase assay revealed that Src kinase induced an about 120% donor/acceptor emission-ratio change of the new biosensor in comparison to 25% change of the original biosensor with the ECFP and Citrine pair. The replacement of Citrine with cpVenus, a circularly permutated version of Venus, which has led to a significant enhancement of a calcium biosensor sensitivity, slightly increased the dynamic range of the Src biosensor to about 40% change. The effect of YPet as a FRET acceptor was further examined in other biosensors. With YPet, the acceptor/donor emission ratio of a Rac biosensor Raichu-Rac with an active mutation V12 (Raichu-RacV12) was 46% higher than that with a negative mutation N17 (Raichu-RacN17). In contrast, it was 28% or 26% when Venus or cpVenus was used for Raichu-Rac, respectively. A significantly enhanced dynamic range of a MT1-MMP FRET biosensor was also observed with ECFP and YPet as the acceptor (570% change before and after MT1-MMP cleavage), compared to 100% with ECFP/Citrine pair and 90% with ECFP/cpVenus pair. ECFP and YPet also led to better sensitivity of a FAK biosensor comparing to the ECFP/Citrine or ECFP/cpVenus pair. The effect of YPet was further analyzed in an ECFP/cpVenus-based calcium biosensor, which has been optimized to achieve a superior dynamic range. A 350% change of cpVenus/ECFP emission ratio was observed between calcium-free and calcium-saturated solutions. When cpVenus was replaced with Citrine, the dynamic range was reduced to 65%, which was restored to 135% when Citrine was replaced with YPet. These results indicate that YPet as the acceptor for FRET can significantly enhance the dynamic range of biosensors for different kinds of molecules, including Src kinase, Rac small GTPase, and MT1-MMP.

### Example 3

### Sensitivity of ECFP and YPet Biosensors in Mammalian Cells

The dynamic ranges of biosensors in mammalian cells were examined. In response to pervanadate (PVD), a tyrosine phosphatase inhibitor and Src kinase activator, the dynamic range of Src ECFP biosensor with YPet as the FRET acceptor was 176% compared to 77% with cpVenus and 32% with Citrine in HeLa cells. When Vav2, a GEF and activator for Rac, was co-expressed together with the Raichu-Rac biosensors in mouse embryonic fibroblasts (MEFs), the YPet-based Raichu-Rac biosensor displayed a 70% increase in emission ratio comparing to 15% with Venus and 5% with cpVenus. Similarly, the MT1-MMP biosensor using ECFP in combination with YPet, but not cpVenus or Citrine, showed a maximal response to 10% fetal bovine serum (FBS) stimulation in HeLa cells. YPet as a FRET acceptor also led to a Ca²⁺ biosensor with much higher sensitivity than Citrine. It is of note that the normalized dynamic range of Ca²⁺ biosensors with YPet was at least similar or even better than that with cpVenus in live HeLa cells (253% vs. 242%). The dynamic range of absolute emission ratio of YPet-based biosensor (scaled at 1-10) was also larger than that of cpVenus (scaled at 0-0.9), allowing a possible enhancement of signal/noise ratio. The dynamic ranges of different biosensors as purified proteins or in live mammalian cells are summarized in Table 2.

TABLE 2

| | Acceptor | | | | | |
|---|---|---|---|---|---|---|
| | | | | Dynamic Range of | | |
| | Dynamic Range of | | | Biosensors in Live | | |
| | Purified Biosensors (%) | | | Cells (%) | | |
| | Citrine | | | Citrine | | |
| ECFP Biosensor | or Venus | cpVenus | YPet | or Venus | cpVenus | YPet |
| Src | 25 | 40 | 120 | 32 | 77 | 176 |
| (donor/acceptor) | | | | | | |
| Rac | 28 | 26 | 46 | 15 | 5 | 70 |
| (acceptor/donor) | | | | | | |
| Ca²⁺ | 65 | 350 | 135 | 41 | 242 | 253 |
| (acceptor/donor) | | | | | | |
| MT1-MMP | 100 | 90 | 570 | 13 | 12 | 50 |
| (donor/acceptor) | | | | | | |

These results indicate that ECFP and YPet can also significantly enhance the dynamic ranges of FRET biosensors in live cells.

### Example 4

### YPet-Based Src Biosensor Activity in Response to VEGF

Both VEGF and Src are critical for angiogenesis. Src has been shown to mediate the effect of VEGF on downstream signaling cascades and cellular functions. However, VEGF-induced Src activation was difficult to detect using an ECFP/Citrine-based biosensor. Ample evidence suggests that Src kinase functions mainly at the cell membrane. Thus, the YPet-based Src biosensor was targeted to the plasma membrane by fusing a prenylation substrate sequence (Lys-Lys-Lys-Lys-Lys-Lys-Ser-Lys-Thr-Lys-Cys-Val-Ile-Met; SEQ ID NO:9) from KRas to visualize the VEGF-induced membrane Src activation in live endothelial cells. VEGF induced a transient Src activation, with a higher activity concentrated at cell periphery. In contrast, the membrane-targeted Src biosensor based on ECFP/Citrine did not show significant FRET change upon VEGF stimulation. Both SU1498, an inhibitor of VEGFR2 (Flk-1), or PP1, an inhibitor of Src, abolished this VEGF-induced FRET response, confirming that the replacement of Citrine by YPet does not alter the biosensor specificity. These results demonstrated that the YPet-based biosensor is more sensitive in detecting the possibly moderate, but physiologically important Src activation in response to VEGF.

### Example 5

### Src Mediates the PDGF-Induced Rac Polarization

The improved ECFP/YPet biosensor was further used in the analysis of molecular hierarchy in cell migration. Both Src and Rac have been shown to play important roles in cell motility and migration. However, the spatiotemporal interrelationship between Src and Rac activation has not been shown. Thus, the improved Rac biosensor was employed to visualize the spatiotemporal Rac activity during cell migration in response to platelet-derived growth factor (PDGF). The role of Src in regulating this Rac activation was also investigated. Mouse embryonic fibroblasts (MEFs) cultured on fibronectin for 3 hours displayed a higher FRET signal of Rac at cell periphery, possibly reflecting the Rac activation induced by the nascent integrin ligation during random cell migration. PDGF induced a strong Rac activation at membrane ruffles and a moderate overall activation of Rac in the whole cell. Both the basal and PDGF-induced Rac activity were significantly inhibited in MEFs pretreated with the Src inhibitor PP1 or in Src/Yes/Fyn triple-knockout (SYF^{-/-}) MEFs. These results suggest that Src is essential for Rac activation. To further characterize the spatiotemporal Rac activity, MEF cells transfected with the Rac biosensor were seeded atop fibronectin-coated stripes (10 μm width) to constrain the cell adhesion and control the direction of cell migration. Rac activity in these cells displayed a moderate polarized distribution, with high activity concentrated at the leading edge. PDGF significantly enhanced this polarization. Again, the inhibition of Src by PP1 or Src/Yes/Fyn triple-knockout blocked the polarized Rac activity before and after PDGF stimulation. Statistical analysis by quantifying the Rac activities over subcellular segments along the migrating direction confirmed the polarized Rac activity, which was significantly enhanced by PDGF and abolished by the inhibition of Src. These results indicate that PDGF induced a polarized Rac activation, which was mediated by Src.

Since Src mediates the polarized Rac activities in response to PDGF, it was subsequently determined whether PDGF also induced a polarized Src activation. Unexpectedly, PDGF-induced Src activation was global without a clear spatial pattern at subcellular levels, even when MEFs were constrained on fibronectin stripes to have directional migration. Interestingly, the PDGF-induced Src activation was inhibited by RacN17, a negative mutant of Rac, but enhanced by RacV12, an active mutant of Rac. These results indicate that although the activations of Src and Rac in response to PDGF have differential spatial characteristics, Src and Rac mutually regulate each other. Further investigations revealed that RacN17 disrupted actin filaments and the disruption of actin filaments by CytoD blocked PDGF-induced Src activation. Together with the observation that Cyto D inhibited PDGF-induced Src activation in MEF cells transfected with RacV12, these results suggest that Rac regulates the PDGF-induced Src via actin cytoskeleton.

### Example 6

### Targeting the MT1-MMP Biosensor to the Plasma Membrane

Since the active domain of MT1-MMP localizes to the extracellular surface of plasma membrane, a cDNA encoding MT1-MMP biosensor was fused with that for the transmembrane domain of PDGF receptor (PDGFR_TM), so that the functional domain of the biosensor which protrudes outward from the surface of plasma membrane is proximal to the catalytic domain of MT1-MMP (FIG. 2). In a cell-based assay, it was confirmed that the MT1-MMP biosensor was targeted to plasma membrane as designed. In fact, an anti-GFP antibody could recognize the membrane-targeted biosensor in cells without permeabilization, but not a cytosolic biosensor without PDGFR_TM.

HeLa cells express minimal endogenous MT1-MMP and hence serve as a good model system to study the functionality of MT1-MMP. EGF can induce a clear FRET change in MT1-MMP-transfected HeLa cells expressing a wild-type biosensor, but not its mutant with the critical cleavage sites Asn-Leu mutated to Ile-Val. The deletion of PDGFR_TM in the biosensor also abolished this EGF-induced FRET response. The results indicate that the EGF-activated MT1-MMP cleaves biosensors at the designed substrate recognition sequence, which occurs at the plasma membrane.

The specificity of the MT1-MMP biosensor was also analyzed using metalloproteinase 2 (TIMP-2), a selective inhibitor of MT1-MMP. In these experiments, HeLa cells were treated with 2.5 μg/ml TIMP-2 for 10 minutes before EGF stimulation. EGF caused a reversed FRET change of the biosensor in cells pretreated with TIMP-2, compared with that observed in non-treated cells. EGF also induced a significant FRET response in HeLa cells co-transfected with MT1-MMP, but not with an empty vector, a negative mutant of MT1-MMP with a Glu240Ala mutation at its catalytic domain (catalytic inactive), a mutant with an ablated transmembrane-domain (cytosolic and inactive), or with MMP-2 or MMP-9. An active mutant of MT1-MMP caused a high ECFP/YPet ratio with or without EGF stimulation. These FRET results were further confirmed by western blot analyses and suggest that the biosensor is specific for detecting MT1-MMP activity in live cells.

### Example 7

### Detection of Circulating Tumor Cells in Breast Cancer

The results presented herein indicate that the FRET-based biosensor disclosed herein is not only useful for detecting MT1-MMP activity in live cells, the present biosensor can also be used to discriminate cancer cells (HT-1080) from normal fibroblasts in culture. However, in so far as the gene transfection efficiency can be low in tissue and biopsy samples, it could be challenging to detect non-transfected cancer cells. Thus, to facilitate binding of the biosensor to a negatively charged cell surface, a polyhistidine peptide with positive charges was fused to the N-terminus of the biosensor. Advantageously, this surface-binding biosensor was found to detect cancer cells in tissue samples. By way of illustration, the gene sequence encoding the biosensor was cloned into PRSETb vector and expressed in BL21 bacterial culture to produce the protein. Fresh tumor and normal tissues were isolated from the same N-methyl-N-nitrosourea (MNU)-induced tumor rats. The incubation of biosensor proteins with these tissue samples led to the observation of an obvious difference in FRET signals between tumor and normal tissues, with the activity of MT1-MMP significantly higher in tumor samples. In contrast, brightfield did not reveal significant difference. Hence, the surface-binding FRET-based biosensor can be applied to detect cancer/tumor cells in tissues.

Membrane type MMPs, in particular, MT1-MMP, play central roles in neoplastic progression and metastasis. In fact, tumor cells coordinate the surface localization of MT1-MMP to digest basement membrane and invade the circulation system. Indeed, the knock-out of MT1-MMP has blocked the capability of tumor cells to penetrate tissues in animal models. Therefore, the activity of MT1-MMP serves as an excellent metastasis biomarker in cancer. Traditional biochemical assays to detect MT1-MMP expression/activity including immunoblotting and zymography require the cells to be killed, wherein the damage caused by the fixation or lysis procedures may result in the alteration or loss of information. In this regard, the instant assay does not require cell lysis or isolated protein analysis and can be used to monitor live cells.

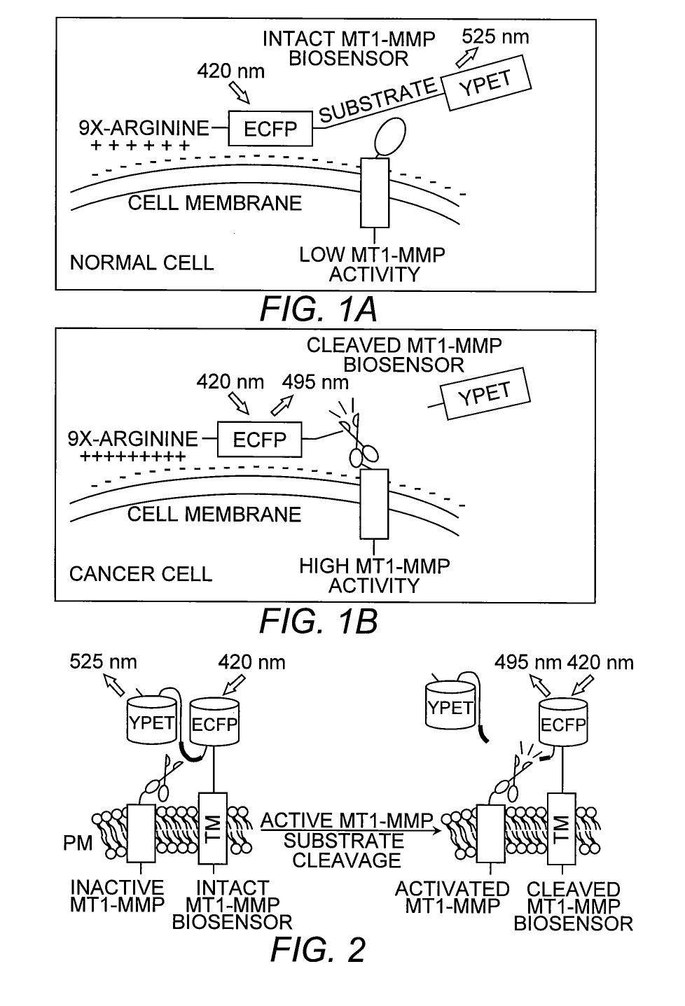

Because CTCs are rare events in blood samples, the efficiency of a biosensor must be sufficient to detect as many CTC as possible. Thus, to further strengthen the binding force between the biosensor and cell surface, the ECFP/YPet biosensor is fused with a positively-charged tag containing nine arginines, which can be tightly absorbed on anionic phospholipids and glycosaminoglycans at the cell surface (FIG. 1). This modified biosensor is used to detect cancer cells in culture under microscopy and in flow cytometry. This modified biosensor is expected to achieve 100% efficiency in illuminating the circulating epithelial cells.

To generate this biosensor, a gene sequence encoding nine arginines is fused in-frame with the cDNA encoding the ECFP/YPet biosensor. The fused gene is subsequently cloned into PRSETb vector and expressed in BL21 bacterial culture to produce the fusion protein. The polyarginine peptide can bind tightly to the cell surface and position the biosensor close to active MT1-MMP. To demonstrate the optimal protein concentrations and incubation time, fibroblast cells are incubated with varying concentrations of the modified biosensor for various periods of time. After the cells are washed with PBS to eliminate the unbound biosensor, they are screened for cyan emission under microscopy to examine binding efficiency. To prevent endocytosis of the biosensor, sodium azide (0.1%) is added into the medium.

Upon the identification of optimal conditions, the modified biosensor protein produced from bacterial culture is incubated with established cancer cells (e.g. HT-1080) or fibroblast cells. The cells are washed with PBS (e.g., three times) and subjected to microscopic observation. The FRET ratio images of HT-1080 and fibroblast cells are acquired and compared between these groups. The biosensor-absorbed HT-1080 and fibroblast cells are also trypsinized and maintained in suspension for flow cytometry screening. The FRET ratios of these two cell types from flow cytometry are compared.

Breast cancer cells are known for their genetic heterogeneity. High MT1-MMP expression or transcription levels have been found in several invasive breast cancer cell lines, such as MDA-231, MDA-435, BT549, HS578T, but not in less invasive cells lines as BT20 and T47-D. Therefore, to establish the threshold of FRET ratio for determining whether a CTC is malignant or not, the FRET ratio of the instant MT1-MMP biosensor can be determined with these known breast cancer cell lines.

Advantageously, flow cytometry can be used to facilitate the high throughput screening of the biosensor FRET ratio in single cells. To confirm that this FRET system can work with flow cytometry, HT-1080 cells are subjected to flow cytometry screening. The flow cytometry system can clearly be used to detect the fluorescent of the cells at CFP and YFP channel, because HT-1080 cells transfected with MT1-MMP biosensor had high fluorescence intensity in both CFP and YFP channel. The data indicated the FRET ratio of CFP/YFP can be visualized in the plot screen. The FRET ratio of transfected HT-1080 appeared to be in a narrow range distinguishable from other populations. The results indicate that the FRET-based MT1-MMP biosensor can be used to detect cancer cell in suspension using the flow cytometry system.

To demonstrate the use of the instant biosensor in flow cytometry screening, human blood samples will be collected from patients who have a breast tumor. Normal blood samples will also be collected from subjects without breast cancer. In brief, peripheral blood (20 ml in EDTA) is obtained from every patient, 3 to 4 weeks after primary surgery and before the initiation of any adjuvant treatment. To avoid contamination with epithelial cells from the skin, all blood samples are obtained at the middle of vein puncture after the first 5 ml of blood were discarded. To separate cells and seminal fluid, the blood samples will be centrifuged 800×g for 15 minutes. The collected cells will be washed three times with PBS containing 0.1 mM EDTA to prevent cell aggregation before they are incubated with biosensor and cell culture medium.

For flow cytometry screening, the biosensor-cell mixture will be centrifuged, washed, and re-suspended in PBS by vortex. The tumor cells with high FRET signals will be counted and separated from those with low FRET signals by flow cytometry. The leukocytes and epithelial cells can be discriminated and sorted based on the cell size indicated by forward scattering signals. The isolated cancer cells will be further confirmed by immunostaining with fluorescence labeled monoclonal antibodies specific for leukocytes (CD45-allophyocyan) and epithelial cancer cells (cytokeratin 8, 18, 19-phycoerythrin). Meanwhile, the non-cancerous cells will also be enriched using magnetic beads and stained on CD-45 and cytokeratins to identify the non-cancerous epithelial cells. As a result, the method of this invention can be compared with a conventional CTC counting assay (CellSearch).

To allow for easier handling of cells and transportation of samples, it can further be tested whether cell fixation can be used in combination with the present biosensor. The cells from blood samples after incubation with biosensors will be washed as described herein and re-suspended in PBS containing 4% paraformaldehyde to fix the cells. These fixed cells with biosensors on surface will be analyzed by flow cytometry for FRET signal detection and immunostaining confirmation for cancer biomarkers. Fixation would allow the shipping of biosensor-labeled cells to core flow cytometry screening centers from local hospitals where flow cytometry is not available.

It is expected that the biosensor will adhere to the surface of all the cells collected from blood. Flow cytometry will be able to distinguish malignant tumor cells from other kinds based on high MT1-MMP activity and FRET signals. It has been reported that paraformaldehye does not affect the properties of fluorescence proteins. Hence, it is also expected that fixation should not affect FRET signals in flow cytometry screening.

Given the importance of MT1-MMP in regulating the metastasis and invasion of the cancer cells, the integration of this biosensor with flow cytometry system will allow an early and convenient method for monitoring and predicting of the progression of breast cancer.

What is claimed is:

1. A isolated biosensor comprising Enhanced Cyan Fluorescent Protein (ECFP) and Yellow fluorescent Protein for Energy Transfer (YPet) operably linked via a membrane-type 1 matrix metalloproteinase recognition sequence.

2. The biosensor of claim 1, further comprising a positively charged tag.

3. The biosensor of claim 2, wherein the tag is a 5 to 30 amino acid residue oligopeptide comprising arginine or histidine.

4. The biosensor of claim 1, further comprising a transmembrane domain.

5. The biosensor of claim 1, wherein the membrane-type 1 matrix metalloproteinase recognition sequence is a 7 to 20 amino acid residue oligopeptide.

6. The biosensor of claim 5, wherein the membrane-type matrix metalloproteinase recognition sequence comprises SEQ ID NO:4, SEQ ID NO:5, SEQ ID NO:6, SEQ ID NO:7, or SEQ ID NO:8.

7. The biosensor of claim 1, wherein said biosensor has a dynamic range of 50% in vivo.

8. An isolated chimeric nucleic acid molecule encoding the biosensor of claim 1.

9. An isolated expression vector harboring the chimeric nucleic acid molecule of claim 8.

10. An isolated host cell containing the expression vector of claim 9.

11. A method for detecting a cancer cell comprising contacting a biological sample with the biosensor of claim 1 and detecting fluorescence resonant energy transfer (FRET), wherein the level of FRET is indicative of the presence of a cancer cell.

12. The method of claim 11, wherein the biological sample is blood.

13. The method of claim 12, wherein the cancer cell is a circulating tumor cell.

14. The method of claim 11, wherein the cancer is metastatic colorectal cancer, breast cancer or prostate cancer.

15. A kit comprising the biosensor of claim 1.
